(19) United States
(12) Patent Application Publication
(10) Pub. No.: US 2022/0346725 A1
(43) Pub. Date: Nov. 3, 2022

(54) VOICE-ASSISTED ACUTE HEALTH EVENT MONITORING

(71) Applicant: Medtronic, Inc., Minneapolis, MN (US)

(72) Inventors: Paul G. Krause, Mahtomedi, MN (US); Christopher D. Koch, Minneapolis, MN (US); Ryan D. Wyszynski, Oak Grove, MN (US); Robert W. Stadler, Shoreview, MN (US); Kevin T. Ousdigian, Shoreview, MN (US); Grant A. Neitzell, Plymouth, MN (US)

(21) Appl. No.: 17/246,362

(22) Filed: Apr. 30, 2021

(51) Int. Cl. A61B 5/00; G06F 40/40

(52) U.S. Cl. CPC A61B 5/7282; A61B 5/0006; A61B 5/0022; A61B 5/6898; A61B 5/681; A61B 5/7405; A61B 5/747; A61B 5/749; G06F 40/40; A61B 2560/02

(57) ABSTRACT

A system comprising processing circuitry configured to receive a wirelessly-transmitted message from a medical device, the message indicating that the medical device detected an acute health event of the patient. In response to the message, the processing circuitry is configured to determine a location of the patient, determine an alert area based on the location of the patient, and control transmission of an alert of the acute heath event of the patient to any one or more computing devices of one or more potential responders within the alert area.

### FIELD

This disclosure generally relates to systems including medical devices and, more particularly, to monitoring of patient health using such systems.

### BACKGROUND

A variety of devices are configured to configured to monitor physiological signals of a patient. Such devices include implantable or wearable medical devices, as well as a variety of wearable health or fitness tracking devices. The physiological signals sensed by such devices include as examples, electrocardiogram (ECG) signals, electroencephalogram (EEG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect acute health events based on the physiological signals, such as episodes of cardiac arrhythmia, myocardial infarction, stroke, or seizure. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events are associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes systems, techniques, and devices for voice-assisted acute health event monitoring. Given the potential to gain important intelligence, the systems, techniques, and devices described herein leverage user input in acute health event monitoring and detection. While a patient's physiological data provides a number of clues regarding the patient's health (both in general and specific to certain class(es) of medical problems), the patient may also provide valuable evidence to consider when evaluating the physiological data (e.g., for additional clues). The systems, techniques, and devices described herein may utilize the patient's input, for example, in new or different rules, to enhance a medical device's acute health event monitoring capabilities.

The present disclosure describes a number of voice or multimedia technologies that the systems, techniques, and devices described herein may avail to obtain the user input. The systems, techniques, and devices described herein may make advantageous use of speech-recognition technologies (e.g., natural language processing), sensors, and networking (e.g., sensor networks) to obtain and then, process the user input.

The present disclosure further describes how employing the systems, techniques, and devices described herein benefits the patient as well as those providing medical assistance to the patient. In some examples where the patient receives acute health monitoring via one or more computing devices (e.g., a mobile device and/or a medical device), the patient may, via vocal responses, answer queries corresponding to an imminent or occurring acute health event (e.g., sudden cardiac arrest) and in turn, the one or more computing devices may provide more effective healthcare. As another benefit, the one or more computing devices may implement a rules engine (e.g., a model such as a mathematical model or a machine learning model) and incorporate the patient's answers in a rules-based evaluation of the patient's physiological data; this is performed instead of using only the patient's physiological data in the rules-based evaluation.

In one example, a computing device comprising: an input device; an output device; processing circuitry; and a memory comprising instructions that, when executed by the processing circuitry, cause the processing circuitry to: determine that sensed physiological data of a patient is indicative of a sudden cardiac arrest of the patient; in response to the determination, and based on the sensed physiological data, generate first audio data configured to cause the output device to output a first plurality of utterances representing a query related to the sudden cardiac arrest; receive second audio data from the input device that represents a second plurality of utterances of at least one of the patient or another user subsequent to the query; and generate output data based on the sensed physiological data and application of natural language processing to the second plurality of utterances.

In another example, a method, by processing circuitry, comprising: determining that sensed physiological data of a patient is indicative of a sudden cardiac arrest of the patient; in response to the determination, and based on the sensed physiological data, generating first audio data configured to cause the output device to output a first plurality of utterances representing a query related to the sudden cardiac arrest; receiving second audio data from the input device that represents a second plurality of utterances of at least one of the patient or another user subsequent to the query; and generating output data based on the sensed physiological data and application of natural language processing to the second plurality of utterances.

In another example, a system comprising processing circuitry configured to: receive a transmission from an implantable medical device indicating that sensed physiological data is indicative of an acute health event for a patient; in response to the transmission, and based on the sensed physiological data, generate first audio data configured to cause the output device to output a first plurality of utterances representing a query related to the acute health event; receive second audio data from the input device that represents a second plurality of utterances of at least one of the patient or another user subsequent to the query; and generate output data based on the sensed physiological data and application of natural language processing to the second plurality of utterances.

In yet another example, a non-transitory computer readable storage medium comprising program instructions configured to cause processing circuitry to: determine that sensed physiological data of a patient is indicative of a sudden cardiac arrest of the patient; in response to the determination, and based on the sensed physiological data, generate first audio data configured to cause the output device to output a first plurality of utterances representing a query related to the sudden cardiac arrest; receive second audio data from the input device that represents a second plurality of utterances of at least one of the patient or another user subsequent to the query; and generate output data based on the sensed physiological data and application of natural language processing to the second plurality of utterances.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to detect acute health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating an example operation by a health monitoring system that operates in accordance with one or more techniques of the present disclosure.

FIG. 6 is a flow diagram illustrating an example operation by a computing device that operates in accordance with one or more techniques of the present disclosure.

Like reference characters refer to like elements throughout the figures and description.

### DETAILED DESCRIPTION

A variety of types of implantable and external devices are configured detect arrhythmia episodes and other acute health events based on sensed ECGs and, in some cases, other physiological signals. External devices that may be used to non-invasively sense and monitor ECGs and other physiological signals include wearable devices with electrodes configured to contact the skin of the patient, such as patches, watches, or necklaces. Such external devices may facilitate relatively longer-term monitoring of patient health during normal daily activities.

Implantable medical devices (IMDs) also sense and monitor ECGs and other physiological signals, and detect acute health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

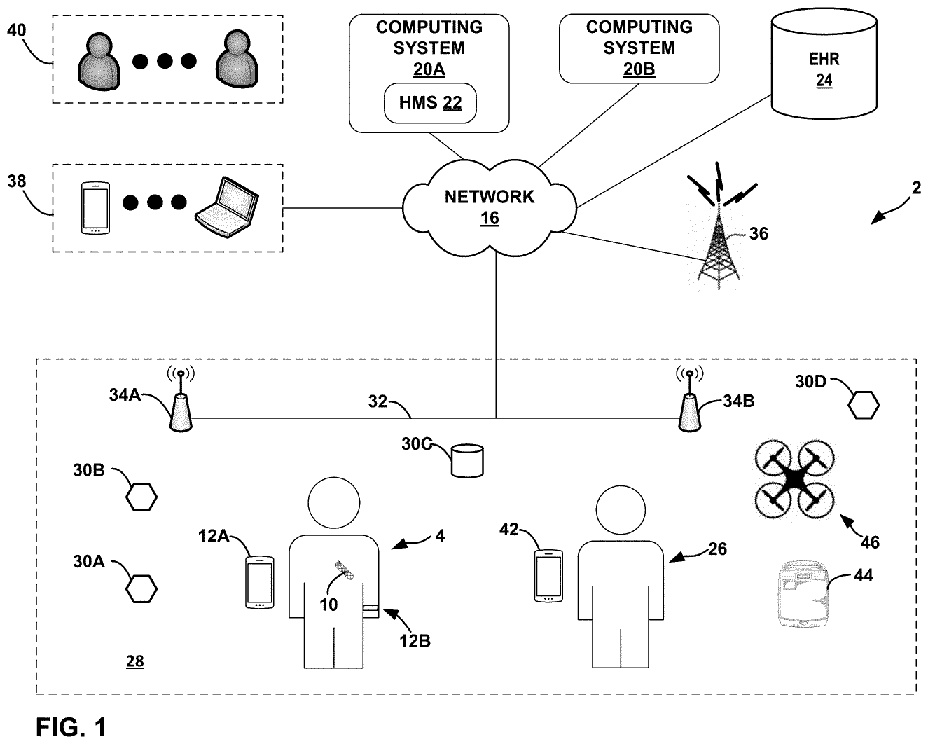

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. The example techniques may be used with one or more patient sensing devices, e.g., IMD 10, which may be in wireless communication with one or more patient computing devices, e.g., patient computing devices 12A and 12B (collectively, “patient computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Patient computing devices 12 are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, tablet computer, or smart television. Computing devices 12 may communicate with IMD 10 and each other according to the Bluetooth® or Bluetooth® Low Energy (BLE) protocols, as examples. In some examples, only one of computing devices 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1A, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilities detection of acute health events of patient 4 by system 2, and the responses of system 2 to such acute health events. HMS 22 may distribute at least some functionality from computing system 20A to device(s) of environment 28.

Computing device(s) 12 may transmit data, including data retrieved from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to detect acute health events of patient 4 based on data sensed by IMD 10 and, in some cases, other data, such as data sensed by computing devices 12A and/or 12B, and data from EHR 24. In response to detection of an acute health event, IMD 10 may wirelessly transmit a message to one or both of computing devices 12A and 12B. The message may indicate that IMD 10 detected an acute health event of the patient. The message may indicate a time that IMD 10 detected the acute health event. The message may include physiological data collected by IMD 10, e.g., data which lead to detection of the acute health event, data prior to detection of the acute health event, and/or real-time or more recent data collected after detection of the acute health event. The physiological data may include values of one or more physiological parameters and/or digitized physiological signals. Examples of acute health events are a cardiac arrest (e.g., a sudden cardiac arrest), a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

In response to the message from IMD 10, computing device(s) 12 may output an alarm that may be visual and/or audible, and configured to immediately attract the attention of patient 4 or any person in environment 28 with patient 4, e.g., a bystander 26. The present disclosure describes alarms as an example of an alert and envisions other example alert types that computing device(s) 12 may generate. Environment 28 may be a home, office, or place of business, or public venue, as examples. Computing device(s) 12 may also transmit a message to HMS 22 via network 16. The message may include the data received from IMD 10 and, in some cases, additional data collected by computing device(s) 12 or other devices in response to the detection of the acute health event by IMD 10. For example, the message may include a location of patient 4 determined by computing device(s) 12. As another example, the message may include input (e.g., vocal responses) from patient 4, for example, patient 4's responses to queries presented by computing device(s) 12 and/or the other devices.

Other devices in the environment 28 of patient 4 may also be configured to output alarms or take other actions to attract the attention of patient 4 and, possibly, a bystander 26, or to otherwise facilitate the delivery of care to patient 4. For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of an acute health event when communication with computing devices 12 is unavailable. In such examples, IoT device(s) 30 may be configured to provide some or all of the functionality ascribed to computing devices 12 herein.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing devices 12, IoT devices 30, and other devices within environment 28 may be configured to communicate with network 16, e.g., with HMS 22, via a cellular base station 36 and a cellular network.

Computing device(s) 12, and in some examples IoT devices 30, may include input devices and interfaces to allow a user to override the alarm in the event the detection of the acute health event by IMD 10 was false. In some examples, one or more of computing device(s) 12 and IoT device(s) 30 may implement an event assistant. The event assistant may provide a conversational interface for patient 4 and/or bystander 26 to exchange information with the computing device or IoT device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user. For example, patient 4 may indicate that they feel dizzy and ask the event assistant, “how am I doing?”.

In some examples, computing device(s) 12 and/or HMS 22 may implement one or more algorithms to evaluate the sensed physiological data received from IMD 10, and in some cases additional physiological or other data sensed or otherwise collected by the computing device(s) or IoT devices 30, to confirm or override the detection of the acute health event by IMD 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event.

In examples in which computing device(s) 12 are configured to perform an acute health event confirmation analysis, computing device(s) 12 may transmit alert messages to HMS 22 and/or IoT devices 30 in response to confirming the acute health event. In some examples, computing device(s) 12 may be configured to transmit the alert messages prior to completing the confirmation analysis, and transmit cancellation messages in response to the analysis overriding the detection of the acute health event by IMD 10. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30.

For example, HMS 22 may be configured to transmit alert messages to one or computing devices 38 associated with one or more care providers 40 via network 16. Care providers 40 may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, and IoT device(s) 30, including sensed physiological data, time of the acute health event, location of patient 4, and results of the analysis by IMD 10, computing device(s) 12, IoT device(s) 30, and/or HMS 22. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or IoT devices 30 may be configured to automatically contact EMS, e.g., autodial 911, in response to receiving an alert message from IMD 10. Again, such operations may be cancelled by patient 4, bystander 26, or another user via a user interface of computing device(s) 12 or IoT device(s) 30, or automatically cancelled by computing device(s) 12 based on a confirmatory analysis performed by the computing device(s) overriding the detection of the acute health event by IMD 10.

Similarly, HMS 22 may be configured to transmit an alert message to computing device 42 of bystander 26, which may improve the timeliness and effectiveness of treatment of the acute health event of patient 4 by bystander 26. Computing device 42 may be similar to computing devices 12 and computing devices 38, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36.

In some examples, the alert message to bystander 26 may be configured to assist a layperson in treating patient. For example, the alert message to bystander 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an automated external defibrillator (AED) 44 or life vest, and instructions for use of the equipment. In some examples, computing device(s) 12, IoT device(s) 30, and/or computing device 42 may implement an event assistant configured to use natural language processing and context data to provide a conversational interface for bystander 42. The assistant may provide bystander 26 with directions for providing care to patient 4, and respond to queries from bystander 26 about how to provide care to patient 4.

In some examples, HMS 22 may mediate bi-directional audio (and in some cases video) communication between care providers 40 and patient 4 or bystander 26. Such communication may allow care providers 40 to evaluate the condition of patient 4, e.g., through communication with patient 4 or bystander 26, or through use of a camera or other sensors of the computing device or IoT device, in advance of the time they will begin caring for the patient, which may improve the efficacy of care delivered to the patient. Such communication may also allow the care providers to instruct bystander 42 regarding first responder treatment of patient 4.

In some examples, HMS 22 may control dispatch of a drone 46 to environment 28, or a location near environment 28 or patient 4. Drone 46 may be a robotic device, such as unmanned aerial vehicle (UAV) or another robot. Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4.

In some examples, HMS 22 may control dispatch of drone 46 (e.g., as an in-home robot) to a home of patient 4. In general, drone 46 may secure the home as a safe environment for patient 4. If emergency care is needed, drone 46 may be equipped to perform certain medical procedures. For example, drone 46 may be configured to access an airway and start ventilation. As another example, drone 46 may move patient 4 away from harm (e.g., by turning off bath water, removing patient from bath, moving patient away a fire or an electrical hazard, opening/unlocking doors, accessing car garage controls, and/or facilitating police and emergency service access to patient 4). HMS 22 may program the robotic device to delivery therapy or recommend therapy based on a cohort analysis of patient 4's current disease state and sensed physiological data. Drone 46 may also be operative to put the patient into a hypothermic state, if needed. In some instances, drone 46 may confirm whether there are witnesses, and activate normal operation if none are detected. Drone 46 may be configured to summon a bystander to support CPR on patient 4. Drone 46 may be configured to make an ECG or pulse measurement or may operate as an AED by touching two parts of patient 4's body with extendable arms having electrodes.

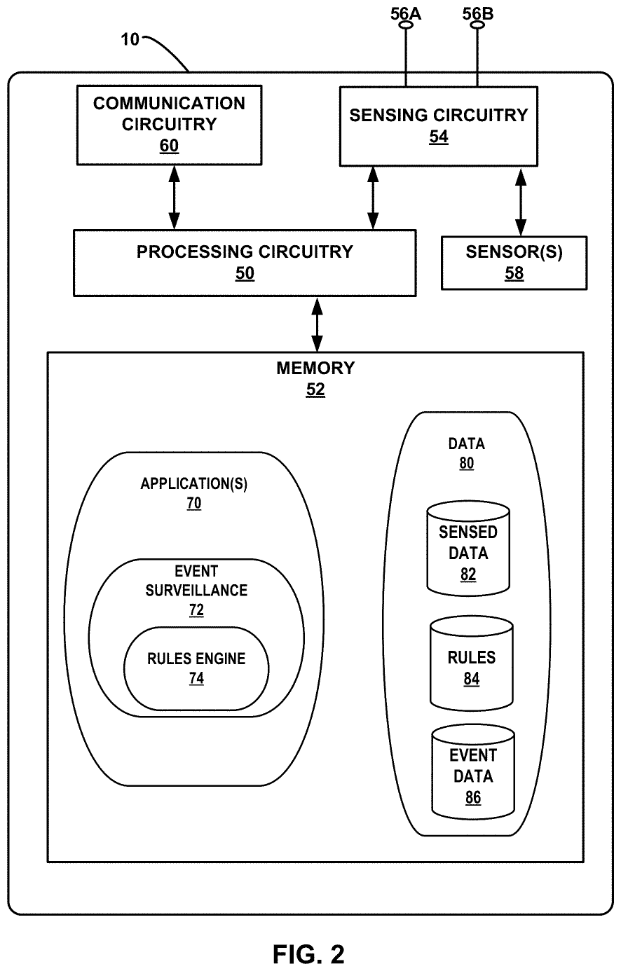

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include an acute health event surveillance application 72. Processing circuitry 50 may execute event surveillance application 72 to detect an acute health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning.

As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart 6 of patient 4 (FIG. 1). In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects an acute health event, event surveillance application 72 may store the sensed data 82 that lead to the detection (and in some cases a window of data preceding and/or following the detection) as event data 86.

In some examples, in response to detection of an acute health event, processing circuitry 50 transmits, via communication circuitry 60, event data 86 for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

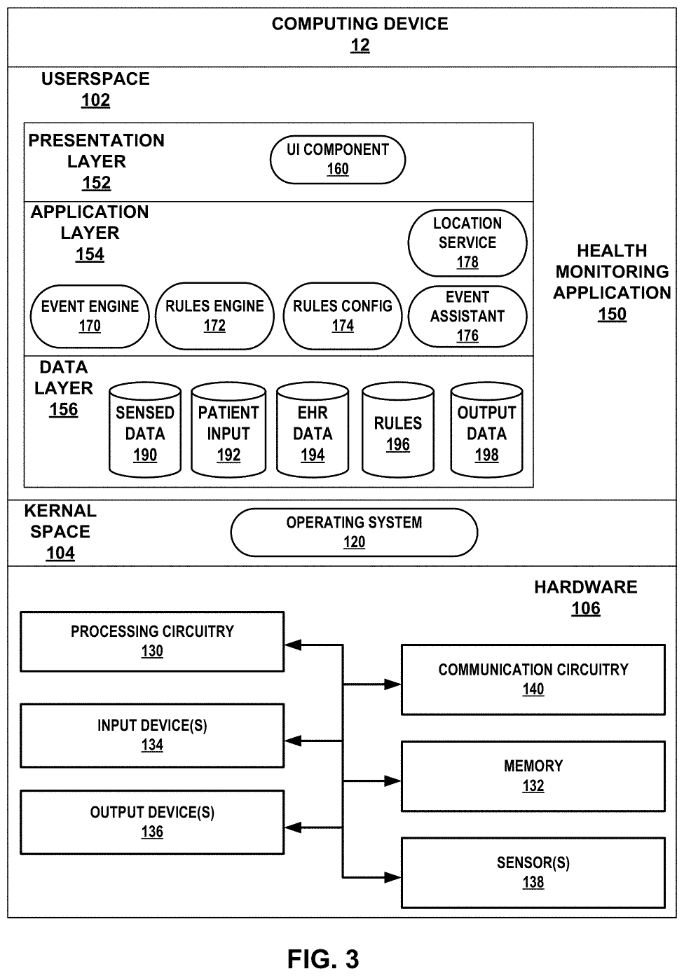

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a smart television, a laptop, a tablet computer, a personal digital assistant (PDA), a smartwatch or other wearable computing device. In some examples, IoT devices 30 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, 3-axis accelerometers, an optical sensor, an impedance sensor, a temperature sensor, a pressure sensor, a heart sound sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 172 may be responsive to receipt of an alert transmission from IMD 10 indicating that IMD 10 detected an acute health event. Event engine 172 may control performance of any of the operations in response to detection of an acute health event ascribed herein to computing device 12, such as activating an alarm, transmitting alert messages to HMS 22, controlling IoT devices 30, and analyzing data to confirm or override the detection of the acute health event by IMD 10.

Rules engine 174 analyzes sensed data 190, and in some examples, patient input 192 and/or EHR data 194, to determine whether there is a sufficient likelihood that patient 4 is experiencing or is about to experience the acute health event detected by IMD 10. Sensed data 190 may include data received from IMD 10 as part of the alert transmission, additional data transmitted from IMD 10, e.g., in “real-time,” and physiological and other data related to the condition of patient 4 collected by computing device(s) 12 and/or IoT devices 30. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystander 26. The queries and responses may occur responsive to the detection of the event by IMD 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Rules engine 172 may apply rules 196 to the data. Rules 196 may include one or more models, algorithms, decision trees, and/or thresholds. In some cases, rules 196 may be developed based on machine learning. In some examples, rules 196 and the operation of rules engine 172 may provide a more complex analysis of the data, e.g., the sensed data received from IMD 10, than is provided by rules engine 74 and rules 84. In some examples, rules 196 include one or more models developed by machine learning, and rules engine 172 applies feature vectors derived from the data to the model(s).

Rules configuration component 174 may be configured to modify rules 196 (and in some examples rules 84) based on feedback indicating whether the detections and confirmations of acute health events by IMD 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

As discussed above, event assistant 176 may provide a conversational interface for patient 4 and/or bystander 26 to exchange information with computing device 12. Event assistant 176 may query the user regarding the condition of patient 4 in response to receiving the alert message from IMD 10. Responses from the user may be included as patient input 192. Event assistant 176 may use natural language processing and context data to interpret utterances by the user. In some examples, the user utterances may confirm (or trigger) that an acute health event occurred, or indicate that an acute health event detected by IMD 10 and/or other devices of system 2 did not occur. A computing device may transmit, forward, or withhold an alert message, or send a cancellation message, based on the utterances. In some examples, processing circuitry of system 2, e.g., of a computing device or HMS 22, may determine a differential diagnosis or appropriate course of care for a detected acute health event based on the utterances. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystander 26.

Location service 178 may determine the location of computing device 12 and, thereby, the presumed location of patient 4. Location service 178 may use global position system (GPS) data, multilateration, and/or any other known techniques for locating computing devices.

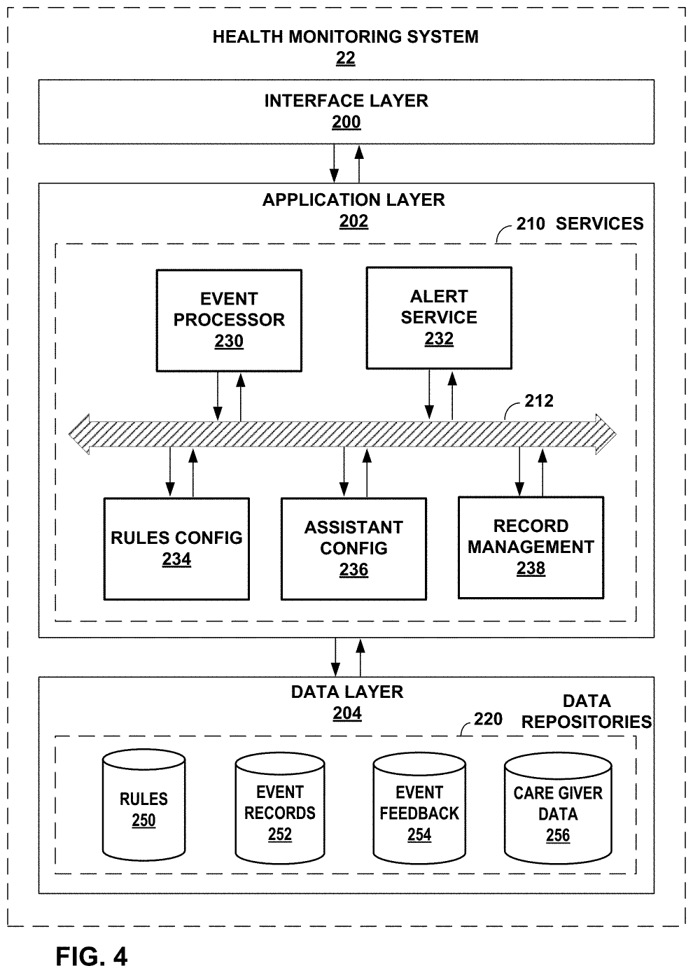

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, bystander 26, and care providers 40, activating drone 46 and, in some cases, analyzing data to confirm or override the detection of the acute health event by IMD 10.

Record management service 238 may store the patient data included in a received alert message within event records 252. Alert service 232 may package the some or all of the data from the event record, in some cases with additional information as described herein, into one more alert messages for transmission to bystander 26 and/or care providers 40. Care giver data 256 may store data used by alert service 232 to identify to whom to send alerts based on locations of potential bystanders 26 and care givers 40 relative to a location of patient 4 and/or applicability of the care provided by care givers 40 to the acute health event experienced by patient 4.

In examples in which HMS 22 performs an analysis to confirm or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMD 10. In such examples, rules configuration service 250 may be configured to develop and maintain rules 196 and rules 84. Rules configuration service 234 may be configured to modify these rules based on event feedback data 254 that indicates whether the detections and confirmations of acute health events by IMD 10, computing device 12, and/or HMS 22 were accurate. Event feedback 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices. For example, assistant configuration service 236 may provide event assistants updates to their natural language processing and context analyses to improve their operation over time. In some examples, assistant configuration service 236 may apply machine learning techniques to analyze sensed data and event assistant interactions stored in event records 252, as well as the ultimate disposition of the event, e.g., indicated by EHR 24, to modify the operation of event assistants, e.g., for patient 4, a class of patients, all patients, or for particular users or devices, e.g., care givers, bystanders, etc.

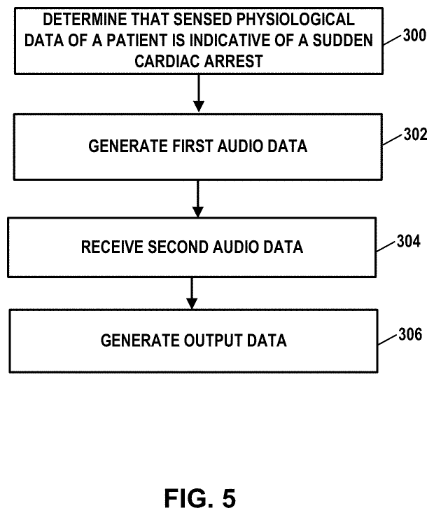

FIG. 5 is a flow diagram illustrating an example operation by a computing device of a health monitoring system that operates in accordance with one or more techniques of the present disclosure. The example operation depicted in FIG. 5 is described with respect to a computing device 12 depicted in FIGS. 1 and 3, but may be described with respect to any computing devices, e.g., computing devices 12, 38, or 42, IoT devices 30, AED 44, drone 46, or health monitoring system (HMS) 22 of FIGS. 1-4 that may implement an event assistant 176.

According to the illustrated example of FIG. 5, processing circuitry of system 2 (e.g., processing circuitry 130 of one or more computing device(s) 12) provides voice-assisted acute health event monitoring based on a patient's physiological data (e.g., sensed data 190) generated by sensing circuitry 52 of IMD 10 and patient input (e.g., patient input 192). According to the illustrated example of FIG. 5, the processing circuitry detects changes in the patient's health caused by an acute health event such as sudden cardiac arrest.

To commence the voice-assisted acute health event monitoring of the example operation of FIG. 5, the processing circuitry determines that sensed physiological data of a patient is indicative of a sudden cardiac arrest (300). In some examples, the processing circuitry makes this determination based on receiving an alert message from another device. In some examples, the processing circuitry analyzes sensed physiological data to determine whether sudden cardiac arrest is detected. As described herein, the processing circuitry may employ a rules engine (e.g., rules engine 74 and/or rules engine 172) to apply various rules (e.g., rules 84 or rules 196) to the sensed physiological data in order to determine whether an acute health event has occurred, is occurring, or is about to occur. In general, each rule sets forth one or more conditions (e.g., minimum or maximum thresholds, ranges, qualities, quantities, and/or the like for parameter values) that, if true, qualify the sensed physiological data as sufficient evidence of an imminent or an occurring acute health event, such as a sudden cardiac arrest.

In response to the determination that the sensed physiological data of the patient is indicative of the sudden cardiac arrest or another acute health event for the patient, the processing circuitry may generate or trigger an alert related to the sudden cardiac arrest or the other acute health event. In some examples, IMD 10 and/or computing device(s) 12 may generate an alert in the form of an auditory alarm and/or or a message communicated to a healthcare provider. In some examples, IMD 10 and/or computer device(s) 12 may trigger the alert by communicating a control directive for a third device to activate an alarm or another alert type. In some examples, IMD 10 and/or computer device(s) 12 may establish a voice or video call with a doctor, a clinician, and/or emergency medical services (EMS).

In (further) response to the determination, and based on the sensed physiological data, the processing circuitry generates first audio data (e.g., output data 198 of FIG. 3) configured to cause the output device to output a first plurality of utterances representing a query related to the sudden cardiac arrest (302). In general, the processing circuitry may configure the first audio data to engage in a conversation with the patient (e.g., to explore what the patient could do) and possibly, provides some closed-loop emotional/humanistic engagement experience. To that end, the first plurality of utterances may be in the form of a question for the patient or another user and include one or more words designed to elicit intelligence, including information that cannot be ascertained from the sensed physiological data (e.g., without difficulty). Any gained intelligence may be incorporated in an application of one or more rules, possibly resulting in a different determination or therapy.

System 2 may employ a number of technologies (e.g., the patient's mobile phone, a smart speaker device, and/or IMD 12 itself) to engage with the patient, for example, to assess the patient's condition. System 2 (e.g., via health monitoring application 150) may generate a vocal representation of the query from the first plurality of utterances. In combination with the vocal representation, the output device may also display, on an electronic display, a textual representation of the first set of utterances, allowing the patient to fully comprehend the query. In some examples, the output device may utilize one or more modes to present the alert of the acute health event, for example, contemporaneous with the first audio data.

In some examples, the query may be in response to initial patient input. For instance, the patient may first inquire “How am I how am I doing?” or state “Virtual Assistant, I am not feeling great”. After the patient's initial input, the patient may proceed to describe current or past symptoms. The processing circuitry may generate the first audio data to present a query related to the acute health event determined from the sensed physiological data and the initial patient input.

The processing circuitry receives from the input device second audio data that represents a second plurality of utterances of at least one of the patient or another user subsequent to the query (304). The second audio data may include the patient's response to the query, including answer(s) to any question(s) regarding the patient's health. After receiving the response from the patient or the other user, the processing circuitry generates output data based on the sensed physiological data and application of natural language processing to the second plurality of utterances. As described herein, the event assistant may employ a number of speech-recognition technologies to perform the natural language processing of the second plurality of utterances.

The first plurality of utterances and the second plurality of utterances may form at least a portion of a conversation between the patient or other user and event assistant 176. The sensed physiological data, as described herein, may be used to evaluate one or more rules for determining whether the patient is experiencing an acute health event. The conversation (particularly, the patient's response) may provide information to evaluate additional or new rules, ensuring a higher level of accuracy in the determination regarding the acute health event. In some instances, as a result of the second plurality of utterances, the processing circuitry may modify a previous determination of the acute health event. The output device, in turn, may generate output data indicative of a modified determination based on the application of natural language processing to the second plurality of utterances. The modified determination may indicate a false rejection, a false detection, a true rejection, or a true rejection of the sudden cardiac arrest. In some examples, based on the application of natural language processing to the second plurality of utterances, the processing circuitry computes or modifies a likelihood of the sudden cardiac arrest based on the sensed physiological data. The processing circuitry of may update a likelihood of the sudden cardiac arrest based on a vocal response from the patient or the other user to the query.

To illustrate by way of example, the output device may utilize one or more modes to present an alarm of sudden cardiac arrest followed by a vocal query such as “How are you feeling?”. As described herein, the patient or another user may provide a response to the vocal query by way of his/her voice and/or via an interface to an input device and that response may proceed to describe symptoms or lack thereof of the sudden cardiac arrest.

To further illustrate, via the output device and/or another device, the processing circuitry may trigger an ongoing alert (e.g., alarm) that as an emergency has occurred and emergence medical services (EMS) is to be summoned. the processing circuitry may communicate a message, via a network connection, to the EMS informing a healthcare provider of the patient's sudden cardia arrest. In other examples, the processing circuitry of may cancel the ongoing alert of the sudden cardiac arrest. In addition to the cancellation of the alarm, the processing circuitry 12 may generate and then, communicate to, one or more healthcare providers, messages conveying that the patient is not experiencing a sudden cardiac arrest or other acute health event. An example message may indicate that the alarm was a false alarm, or that the sudden cardiac arrest determination is a true detection but that the patient has recovered or is stable. An example message may prompt a user or device to alter the patient's therapy (e.g., to start/stop CPR). Another example message may prompt a user or device to remove a specific treatment. Another example message may be communicated to the EMS in order to redirect an ambulance to a specialized care center or another caregiver. Another example message may be communicated to a user or device 2 to trigger a specific treatment (for example, instruct the patient or bystander 42 to utilize an AED, find the location of a nearby drone-delivered AED, take medication or another treatment, and/or the like) and then, add that specific treatment to the patient's therapy.

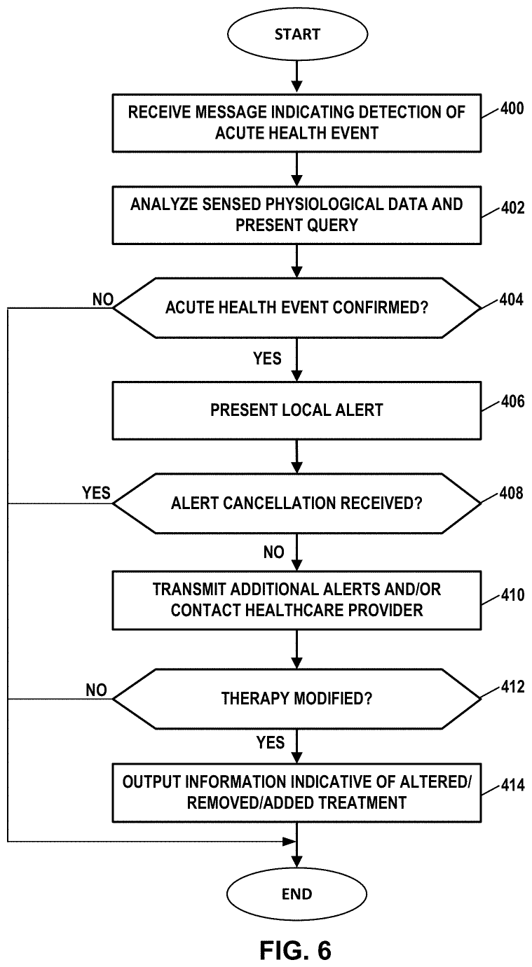

FIG. 6 is a flow diagram illustrating an example operation by computing device(s) 12 (that operates in accordance with one or more techniques of the present disclosure. Although described with respect to computing device(s) 12, the example of FIG. 6 may be performed by any one or more of computing devices 12, 38 or 42, IoT devices 30, AED 44, or drone 46. The example operation depicted in FIG. 6 may be described with respect to system 2 of FIGS. 1-4.

Computing device(s) 12 may include a mobile device (e.g., a smartphone or smartwatch), a desktop computer, or a smart television. According to the illustrated example of FIG. 6, processing circuitry 130 of computing device 12, e.g., via event assistant 176 of health monitoring application 150, provides voice-based acute health event monitoring and detection and, in some instances, employs a patient's mobile device or a smart speaker to output audio data for queries.

Processing circuitry 130 of computing device(s) 12 may receive a message indicating detection of acute health event (400). In some examples, computing device 12 generates an alert, for instance, by activating an audible or visual alarm. Processing circuitry 130 of computing device(s) 12 may analyze the patient's sensed physiological data and present a query to that patient (402), for example, to confirm or reject the detection of the acute health event. Processing circuitry 130 of computing device(s) 12 may execute instructions to determine whether to confirm the acute health event based on the analysis, including responses to the queries (404).

As described herein, processing circuitry 130 may employ a rules engine 172 to render a determination (e.g., a likelihood) regarding whether the sensed physiological data includes sufficient evidence for the acute health event. Processing circuitry 130 of computing device(s) 12 may determine one or more queries (e.g., a first query and a next query after the patient's response) based on one or more rules such that the patient is presented with at least one question configured to elicit information relevant to applying the one or more rules. The patient's response may indicate information that processing circuitry 130 of computing device(s) 12 may leverage in rendering a more accurate determination of the acute health event.

Based on a determination that the acute health event cannot be confirmed (NO of 404), processing circuitry 130 of computing devices 12 not present, or may terminate a local alert, and the processes may end. In some examples, processing circuitry 130 of computing devices 12 may update a likelihood computed for the acute health event to account for the patient's response to the query. With respect to the initial detection of the acute health event as mentioned above, processing circuitry 130 of computing devices 12 generates a modified determination indicating a false detection. Based on a determination that the acute health event can be confirmed (YES of 404), processing circuitry 130 of computing devices 12 may continue the local alert as an ongoing alert (406).

Processing circuitry 130 of computing device(s) 12 may proceed to execute instructions to determine whether an alert cancelation has been received, e.g., based on user input (408). Based on a determination that the cancellation has not been received (NO of 408), processing circuitry 130 of computing device(s) 12 transmits additional alerts and/or contacts a healthcare provider, such as EMS (410). Based on a determination that the cancellation has been received (YES of 408), processing circuitry 130 of computing devices 12 may continue the local alert as an ongoing alert (END).

Processing circuitry 130 of computing device(s) 12 may proceed to execute instructions to determine whether to modify the patient's therapy (412). In some examples, processing circuitry 130 of computing device(s) 12 performs a determination regarding whether to alter, add, or remove any specific treatments. In other examples, IMD 12 may perform the determination regarding whether to alter, add, or remove any specific treatments, for instance, in response to a confirmation of the acute health event. Based on a determination that the therapy is to be modified (YES of 412), processing circuitry 130 of computing device(s) 12 outputs information indicative of an altered (first) treatment, a removed (second) treatment, and/or an additional (third) treatment (414). Based on a determination that the therapy is not to be modified (NO of 412), processing circuitry 130 of computing device(s) 12 may continue the local alert as an ongoing alert (END).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Example 1: A computing device includes an input device; an output device; processing circuitry; and a memory includes determine that sensed physiological data of a patient is indicative of an acute health event of the patient; in response to the determination, and based on the sensed physiological data, generate first audio data configured to cause the output device to output a first plurality of utterances representing a query related to the acute health event; receive second audio data from the input device that represents a second plurality of utterances of at least one of the patient or another user subsequent to the query; and generate output data based on the sensed physiological data and application of natural language processing to the second plurality of utterances.

Example 2: The computing device of example 1, wherein to generate the output data, the instructions cause the processing circuitry to: generate output data indicative of a modified determination based on the application of natural language processing to the second plurality of utterances, wherein the modified determination comprises a false rejection or a false detection of the acute health event.

Example 3: The computing device of any of examples 1 and 2, wherein to determine that the sensed physiological data of the patient is indicative of the acute health event of the patient, the instructions cause the processing circuitry to: in response to the determination, generate or trigger activation of an alert related to the acute health event.

Example 4: The computing device of any of examples 1 through 3, wherein to generate the output data, the instructions cause the processing circuitry to: based on the application of natural language processing to the second plurality of utterances, determine whether to cancel or alter an alert for the acute health event.

Example 5: The computing device of any of examples 1 through 4, wherein the memory further comprises instructions that, when executed by the processing circuitry, cause the processing circuitry to: based on the application of natural language processing to the second plurality of utterances, determine whether to modify therapy delivery to the patient by at least one of adding, modifying, or removing at least one treatment.

Example 6: The computing device of any of examples 1 through 5, wherein to generate the output data, the instructions cause the processing circuitry to: based on the application of natural language processing to the second plurality of utterances and the sensed physiological data, compute a likelihood of the acute health event.

Example 7: The computing device of any of examples 1 through 6, wherein to generate the first audio data, the instructions cause the processing circuitry to: determine the query based on at least one of the sensed physiological data or an initial patient input.

Example 8: The computing device of any of examples 1 through 7, wherein to generate the output data, the instructions cause the processing circuitry to: update a likelihood of the acute health event based on a vocal response from the patient or the other user to the query.

Example 9: The computing device of any of examples 1 through 8, wherein to receive the second audio data, the instructions cause the processing circuitry to: determine a next query based on the second audio data in response to the query.

Example 10: The computing device of any of examples 1 through 9, wherein to generate the first audio data, the instructions cause the processing circuitry to: generate the query to provide data for a rule of a rules engine configured to detect acute health event occurrences from the sensed physiological data.

Example 11: The computing device of any of examples 1 through 10, wherein, to determine that sensed physiological data of a patient is indicative of an acute health event of the patient, the instructions cause the processing circuitry to: receive a transmission from an implantable medical device indicating that the sensed physiological data is indicative of acute health event.

Example 12: The computing device of any of examples 1 through 11, wherein, to determine that sensed physiological data of a patient is indicative of an acute health event of the patient, the instructions cause the processing circuitry to: receive a transmission from an implantable medical device including the sensed physiological data; and determine that the sensed physiological data is indicative of acute health event.

Example 13: The computing device of any of examples 1 through 12, wherein the computing device comprises at least one of a smartphone, a smartwatch, a smart speaker, or a smart television.

Various examples have been described. These and other examples are within the scope of the following claims.

1. A computing device comprising:
an input device;
an output device;
processing circuitry; and
a memory comprising instructions that, when executed by the processing circuitry, cause the processing circuitry to:
determine that sensed physiological data of a patient received from an insertable cardiac monitor implanted in the patient indicates that the patient is experiencing or will imminently experience a sudden cardiac arrest;
in response to the determination, while confirming whether the patient is experiencing or will imminently experience the sudden cardiac arrest, and based on the sensed physiological data, generate, by an event assistant executing at the processing circuitry that provides a conversational interface between the computing device and at least one of the patient or a bystander who is proximate in location to the patient to evaluate a condition of the patient, first audio data configured to cause the output device to output a first plurality of utterances representing a query related to the sudden cardiac arrest that prompts at least one of the patient or the bystander to respond with a natural language spoken response;
receive second audio data from the input device that represents a second plurality of utterances of at least one of the patient or the bystander subsequent to the query while confirming whether the patient is experiencing or will imminently experience the sudden cardiac arrest;
apply natural language processing to determine an interpretation of the second plurality of utterances as natural language spoken by the at least one of the patient or the bystander; and
generate, while confirming whether the patient is experiencing or will imminently experience the sudden cardiac arrest, output data based on the sensed physiological data and the interpretation of the second plurality of utterances as natural language spoken by the at least one of the patient or the bystander.

2. The computing device of claim 1, wherein to generate the output data, the instructions cause the processing circuitry to:
generate output data indicative of a modified determination based on the interpretation of the second plurality of utterances, wherein the modified determination comprises a false rejection or a false detection of the sudden cardiac arrest.

3. The computing device of claim 1, wherein to determine that the sensed physiological data of the patient is indicative of the sudden cardiac arrest of the patient, the instructions cause the processing circuitry to:
in response to the determination, generate or trigger activation of an alert related to the sudden cardiac arrest.

4. The computing device of claim 1, wherein to generate the output data, the instructions cause the processing circuitry to:
based on the interpretation of the second plurality of utterances, determine whether to cancel or alter an alert for the sudden cardiac arrest.

5. The computing device of claim 1, wherein the memory further comprises instructions that, when executed by the processing circuitry, cause the processing circuitry to:
based on the interpretation of the second plurality of utterances, determine whether to modify therapy delivery to the patient by at least one of adding, modifying, or removing at least one treatment.

6. The computing device of claim 1, wherein to generate the output data, the instructions cause the processing circuitry to:
based on the interpretation of the second plurality of utterances and the sensed physiological data, compute a likelihood of the sudden cardiac arrest.

7. The computing device of claim 1, wherein to generate the first audio data, the instructions cause the processing circuitry to:
determine the query based on at least one of the sensed physiological data or an initial patient input.

8. The computing device of claim 1, wherein to generate the output data, the instructions cause the processing circuitry to:
update a likelihood of the sudden cardiac arrest based on the interpretation of the second plurality of utterances as natural language spoken by the at least one of the patient or the other user in response to the query.

9. The computing device of claim 1, wherein to receive the second audio data, the instructions cause the processing circuitry to:
determine a next query based on the interpretation of the second plurality of utterances as natural language spoken by the at least one of the patient or the other user in response to the query.

10. The computing device of claim 1, wherein to generate the first audio data, the instructions cause the processing circuitry to:
generate the query to provide data for a rule of a rules engine configured to detect sudden cardiac arrest occurrences from the sensed physiological data.

11. The computing device of claim 1, wherein, to determine that the sensed physiological data of a patient indicates that the patient is experiencing or will imminently experience a sudden cardiac arrest, the instructions cause the processing circuitry to:
receive a transmission from the insertable cardiac monitor indicating that the sensed physiological data is indicative of sudden cardiac arrest.

12. The computing device of claim 1, wherein, to determine that the sensed physiological data of a patient indicates that the patient is experiencing or will imminently experience a sudden cardiac arrest of the patient, the instructions cause the processing circuitry to:
receive a transmission from the insertable cardiac monitor including the sensed physiological data; and
determine that the sensed physiological data is indicative of the sudden cardiac arrest.

13. The computing device of claim 1, wherein the computing device comprises at least one of a smartphone, a smartwatch, a smart speaker, or a smart television.

14. A method comprising, by processing circuitry:
determining that sensed physiological data of a patient received from an insertable cardiac monitor implanted in the patient indicates that the patient is experiencing or will immanently experience a sudden cardiac arrest;
in response to the determination, while confirming whether the patient is experiencing or will imminently experience the sudden cardiac arrest, and based on the sensed physiological data, generating, by an event assistant executing at the processing circuitry that provides a conversational interface between a computing device and at least one of the patient or a bystander who is proximate in location to the patient to evaluate a condition of the patient, first audio data configured to cause an output device to output a first plurality of utterances representing a query related to the sudden cardiac arrest that prompts at least one of the patient or the bystander to respond with a natural language spoken response;
receiving second audio data from an input device that represents a second plurality of utterances of at least one of the patient or the bystander subsequent to the query while confirming whether the patient is experiencing or will imminently experience the sudden cardiac arrest;
apply natural language processing to determine an interpretation of the second plurality of utterances as natural language spoken by the at least one of the patient or the bystander; and
generating, while confirming whether the patient is experiencing or will imminently experience the sudden cardiac arrest, output data based on the sensed physiological data and the interpretation of the second plurality of utterances as natural language spoken by the at least one of the patient or the bystander.

15. The method of claim 14, wherein generating the output data comprises:
generating output data indicative of a modified determination based on the interpretation of the second plurality of utterances, wherein the modified determination comprises a false rejection or a false detection of the sudden cardiac arrest.

16. The method of claim 14, wherein generating the output data further comprises:
in response to the determination, generating or triggering activation of an alert related to the sudden cardiac arrest.

17. The method of claim 14, wherein generating the output data further comprises:
based on the interpretation of the second plurality of utterances, determining whether to cancel or alter an alert for the sudden cardiac arrest.

18. The method of claim 14, wherein generating the output data further comprises:
based on the interpretation of the second plurality of utterances, determining whether to modify therapy delivery to the patient by at least one of adding at least one treatment, modifying at least one second treatment, or removing at least one third treatment.

19. The method of claim 14, wherein generating the output data further comprises:
based on the interpretation of the second plurality of utterances and the sensed physiological data, computing a likelihood of the sudden cardiac arrest.

20. The method of claim 14, wherein generating the first audio data further comprises:
determining the query based on at least one of the sensed physiological data or an initial patient input.

21. The method of claim 14, wherein generating the output data further comprises:
updating a likelihood of the sudden cardiac arrest based on the interpretation of the second plurality of utterances as natural language spoken by the at least one of the patient or the other user in response to the query.

22. The method of claim 14, wherein receiving the second audio data further comprises:
determining a next query based on the interpretation of the second plurality of utterances as natural language spoken by the at least one of the patient or the other user in response to the query.

23. The method of claim 14, wherein generating the first audio data further comprises:
generating the query to provide data for a rule of a rules engine configured to detect sudden cardiac arrest occurrences from the sensed physiological data.

24. The method of claim 14, wherein determining that the sensed physiological data of a patient indicates that the patient is experiencing or will imminently experience a sudden cardiac arrest further comprises:
receiving a transmission from the insertable cardiac monitor indicating that the sensed physiological data is indicative of sudden cardiac arrest.

25. The method of claim 14, wherein determining that the sensed physiological data of a patient indicates that the patient is experiencing or will imminently experience a sudden cardiac arrest further comprises:
receiving a transmission from the insertable cardiac monitor including the sensed physiological data; and
determining that the sensed physiological data is indicative of the sudden cardiac arrest.

26. The method of claim 14, wherein the output device or the input device comprises at least one of a smartphone, a smartwatch, a smart speaker, or a smart television.

27. A system comprising processing circuitry configured to:
receive a transmission from an insertable cardiac monitor indicating that sensed physiological data indicates that the patient is experiencing or will immanently experience an acute health event;
in response to the transmission, while confirming whether the patient is experiencing or will imminently experience the acute heath event, and based on the sensed physiological data, generate, by an event assistant executing at the processing circuitry that provides a conversational interface between a computing device and at least one of the patient or a bystander who is proximate in location to the patient to evaluate a condition of the patient, first audio data configured to cause the output device to output a first plurality of utterances representing a query related to the acute health event that prompts at least one of the patient or the bystander to respond with a natural language spoken response;
receive second audio data from the input device that represents a second plurality of utterances of at least one of the patient or the bystander subsequent to the query while confirming whether the patient is experiencing or will imminently experience the acute health event;
apply natural language processing to determine an interpretation of the second plurality of utterances as natural language spoken by the at least one of the patient or the bystander; and
generate, while confirming whether the patient is experiencing or will imminently experience the acute health event, output data based on the sensed physiological data and the interpretation of the second plurality of utterances as natural language spoken by the at least one of the patient or the bystander.

28. The system of claim 27, wherein the processing circuitry is configured to:
receive a transmission from the insertable cardiac monitor including the sensed physiological data; and
determine that the sensed physiological data is indicative of the acute health event.

29. The system of claim 27, wherein the processing circuitry is configured to:
in response to the determination, generate or trigger activation of an alert related to the acute health event.

30. A non-transitory computer readable storage medium comprising program instructions configured to cause processing circuitry to:
determine that sensed physiological data of a patient received from an insertable cardiac monitor is indicative of a sudden cardiac arrest of the patient;
in response to the determination, while confirming whether the patient is experiencing or will imminently experience the sudden cardiac arrest, and based on the sensed physiological data, generate, by an event assistant executing at the processing circuitry that provides a conversational interface between a computing device and at least one of the patient or a bystander who is proximate in location to the patient to evaluate a condition of the patient, first audio data configured to cause the output device to output a first plurality of utterances representing a query related to the sudden cardiac arrest that prompts at least one of the patient or the bystander to respond with a natural language spoken response;
receive second audio data from the input device that represents a second plurality of utterances of at least one of the patient or the bystander subsequent to the query while confirming whether the patient is experiencing or will imminently experience the sudden cardiac arrest;
apply natural language processing to determine an interpretation of the second plurality of utterances as natural language spoken by the at least one of the patient or the bystander; and
generate, while confirming whether the patient is experiencing or will imminently experience the sudden cardiac arrest, output data based on the sensed physiological data and the interpretation of the second plurality of utterances as natural language spoken by the at least one of the patient or the bystander.
